(19) United States
(12) Patent Application Publication
(10) Pub. No.: US 2006/0002922 A1
(43) Pub. Date: Jan. 5, 2006

(54) Method for preventing or treating obesity by modulating the activities of the pentose phosphate patway

(75) Inventor: Zhengping Xu, Gaithersburg, MD (US)

Correspondence Address: CROWELL & MORING LLP;INTELLECTUAL PROPERTY GROUP, P.O. BOX 14300, WASHINGTON, DC, 20044-4300, US

(73) Assignee: Minkon Biotechnology, Inc., Gaithersburg, MD (US)

(21) Appl. No.: 11/169,944

(22) Filed: Jun. 30, 2005

Related U.S. Application Data
(60) Provisional application No. 60/583,617, filed on Jun. 30, 2004.

(51) Int. Cl. A61K 48/00; A61K 39/395

(52) U.S. Cl. 424/141.1; 514/44

(57) ABSTRACT

Method for preventing or treating overweight or obesity, the method comprising administering to a patient in need thereof an antagonist that inhibits gene expression or activity of an enzyme that is involved or related to the pentose phosphate pathway. Preferably, the enzyme is a transketolase, and the antagonist is an antibody, an antisense molecule, an siRNA molecule, a molecule for forming a triplex nucleic acid molecule with the enzyme-encoding polynucleotide. Also disclosed are pharmaceutical compositions comprising the same, and method for screening a substance that inhibits gene expression of a PPP enzyme or its function.

### CROSS REFERENCE TO RELATED APPLICATIONS

This application claims the priority of U.S. Provisional Application No. 60/583,617, filed Jun. 30, 2004, the disclosure of which is expressly incorporated by reference herein.

### BACKGROUND OF THE INVENTION

In the United States and in much of the developed world, obesity has risen at an epidemic rate during the past 20 years. According to the Centers for Disease Control, the situation is worsening rather than improving. In the United States, in 1991, only four states were reporting obesity prevalence rates of 15-19 percent and no states reported rates at or above 20 percent. In 2002, 20 states have obesity prevalence rates of 15-19 percent; 29 states have rates of 20-24 percent; and one state reports a rate over 25 percent.

Obesity is defined as an excessively high amount of body fat or adipose tissue in relation to lean body mass. (Stunkard A J, Wadden T A. (Editors) Obesity: theory and therapy, Second Edition. New York: Raven Press, 1993, at p 14; National Research Council. Diet and health: implications for reducing chronic disease risk. Washington, D.C.: National Academy Press, 1989, at p 114). The amount of body fat (or adiposity) includes concern for both the distribution of fat throughout the body and the size of the adipose tissue deposits. Body mass index (BMI) is a common measure expressing the relationship (or ratio) of weight-to-height. It is a mathematical formula in which a person's body weight in kilograms is divided by the square of his or her height in meters (i.e., wt/(ht)₂. The BMI is more highly correlated with body fat than any other indicator of height and weight (NRC p563). Individuals with a BMI of 25 to 29.9 are considered overweight, while individuals with a BMI of 30 or more are considered obese.

Overweight and obese individuals (BMI of 25 and above) are at increased risk for physical ailments such as high blood pressure, hypertension, high blood cholesterol, dyslipidemia, type 2 (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence), uric acid nephrolithiasis, psychological disorders (such as depression, eating disorders, distorted body image, and low self esteem).

One of the national health objectives for the United States for the year 2010 is to reduce the prevalence of obesity among adults to less than 15%.

Obesity is generally considered to be a metabolic abnormality caused by imbalanced energy intake and expenditure (Kopelman, 2000; Spiegelman et al., 2001). Genetic susceptibility plus environmental factors are both contributing to the rapid rise of obesity population. It has been believed that the high prevalence of obesity in the last twenty years is largely due to environmental changes, such as the availability and composition of food (Jequier, 2002; Swinburn, et al., 2002). However, the exact mechanisms by which environmental factors affect the metabolic pathways that directly or indirectly impact obesity are largely unknown at present. A high fat diet has been considered to be responsible for the increased weight gain (Hill, et al., 2000; Peters, 2003; Masuzaki, et al., 2003). Dietary fats alone, however, most probably are not the only factor that contribute to the high prevalence of excess body fat in modern society (Willett, 2002).

A variety of methods are commonly used for treating obesity, including non-pharmacotherapy methods, such as behavior, surgery, diet restriction and exercising. However, other than surgical methods, these non-pharmacotherapy methods have proven to have only limited or short-term effects on weight loss. These methods are especially unacceptable for severe obesity, age-related obesity or obesity with co-morbidity diseases. For example, dietary therapy poses the risk of depriving the patient of necessary nutrients, while it is simply a formidable challenge to lose 50 to 100 pounds of body weight by exercise. These patients usually turn to the help of surgical treatment.

Surgical methods basically use a mechanical procedure to force the reduction of body fat. Gastric bypass is a common procedure used to make the stomach smaller. The primary objectives of these procedures are to decrease food intake and/or cause mal-absorption. Surgical procedures are expensive, and engenders patient suffering during and after the operation. Furthermore, many adverse side effects exist (Bell, 2004; Brody, 2004; Colquitt, et al., 2003). For example, 10 to 25% patients who have weight-loss operations require follow up operations or secondary surgery, and more than one-third of them develop gallstones (Everhart, 1993). However, due to the lack of effective alternative methods for weight loss, especially the low efficacy and scarcity of anti-obesity drugs in the current market, surgical intervention is the only choice for many people, such as those who are severely obese, or obese with co-morbidity diseases.

Although pharmacotherapy is preferred for obesity treatment, the development of anti-obesity drugs has not been very successful, due to low efficacy and unacceptable side effects (Glazer, 2001; Beermann, et al., 2001). Some of the weight control drugs that have hit the market carry the risk of unpleasant or even life-threatening side effects (Haller, 2002). Others have proven to be only partially effective, as they also involve harsh dietary regimes that patients can hardly follow for long (Nightingale, 1993; Bray, 1993).

Accordingly, there is a great need for novel methods and pharmaceutical compositions that can be used to prevent or treat overweight or obesity.

Fatty acid synthesis was shown to be increased in liver and adipose tissue of obese mice (Hems, et al., 1975). Several reports indicate that adipose tissue might be an important de novo lipogenic site and might contribute significantly to the increased fat in obese patients (Larsen, 2002; Diraison, et al., 2002). Regardless, the conventional wisdom is that in humans, endogenous fatty acid synthesis, which takes place mainly in the liver (Gandemier, et al., 1982; Patel, et al., 1975), has only a minor contribution to human obesity (Guo, et al., 2000; Hellerstein, et al., 1991), and dietary fat is considered as the main nutrient contributing to the increased body fat in human.

### BRIEF DESCRIPTION OF THE DRAWINGS

FIG. 1 shows the body weight gain of four groups of mice fed with a high fat diet with and without an TKT inhibitor oxythamine.

FIG. 2 shows that mice fed with normal diet (D12450) (G1a and G1b) and mice fed with high fat diet (D12451) alone (G2a and G2b), gained most weight. In contrast, mice fed with high fat diet plus FWGE (G7a and G7b), and mice fed with normal diet plus FWGE (G9a and G9b) gained less weight.

### DESCRIPTION OF THE INVENTION

Recently, research appears to suggest that carbohydrate could produce high glycemic response, resulting in higher carbohydrate oxidation at the expense of fat oxidation and thus promoting a net body fat gain (Centers for Disease Control and Prevention (CDC), 2001; MMWR, 2003; Minehira, et al., 2003). Dietary sugar and/or carbohydrates might play much broader roles in the regulation of body fat besides its role of reducing fat oxidation and thus favoring fat storage in adipose tissue (Levine, et al., 2003; Matsuo, et al., 2001; Foster, et al., 2003; Parks, et al., 2002).

For the first time, the present inventors discovered that modulating the pentose phosphate pathway (PPP) activity, especially modulating the expression or activity of the enzyme transketolase, a key enzyme in the nonoxidative reactions of the PPP, can be used to prevent or treat obesity or overweight. Accordingly, the present invention provides methods and pharmaceutical composistions that are suitable for treating or preventing, or both, overweight or obesity by modulating the activities of the pentose phosphate pathway (PPP). In a preferred embodiment, the present invention provides methods and compositions that modulate the level or activity of transketolase (TKT).

The pentose phosphate pathway (PPP), also called the phosphogluconate pathway, is situated between glycolysis and a variety of different biosynthetic cascades, and provides D-erythrose 4-phosphate from the pathway's non-oxidative branch as the carbon source to the common aromatic pathway. The non-oxidative pentose phosphate pathway converts D-fructose 6-phosphate into varying equivalents of D-ribose 5-phosphate, D-sedoheptulose 7-phosphate, and D-erythrose 4-phosphate which are required, respectively, for the biosynthesis of nucleotides, gram-negative bacterial lipopolysaccharides, and compounds such as the aromatic amino acids phenylalanine, tyrosine, and tryptophan derived from the common aromatic biosynthetic pathway. Nonoxidative pentose phosphate pathway interconversion between D-fructose 6-phosphate and pentoses allows organisms such as E. coli to use the pentoses D-ribose, D-xylose, and D-arabinose as exclusive sources of carbon during growth.

The PPP also generates sugar phosphates for intermediary biosynthesis and nucleic acid synthesis and NADPH for reductive biosynthesis (Schenk et al., 1998; Wood, 1985, The pentose phosphate pathway. Academic Press, New York, N.Y.). Approximately 40% of NADPH is generated by pentose phosphate pathway. NADPH carries chemical energy in the form of reducing power and is used almost universally as the reductant in anabolic pathways.

In mammals, this role of NADPH, and thus the activity of the PPP, is especially prominent in tissues actively synthesizing fatty acids and steroids, because fatty acid biosynthesis and steroid biosynthesis utilize large amounts of NADPH. These tissues include mammary gland, adrenal cortex, liver, and adipose tissue. These tissues use NADPH to reduce the double bonds and carbonyl groups in intermediates in the synthetic process. Tissues less active in synthesizing fatty acids, such as skeletal muscle, are virtually lacking in activities of the PPP.

According to the present invention, each and every component of the PPP, and enzymes that are directly related to the PPP can be modulated as a method to prevent or treat overweight or obesity. These enzymes include those involved in the oxidative reactions of the PPP (e.g. glucose 6-phosphate dehydrogenase, lactonase, 6-phosphogluconate dehydrogenase, and phosphopentose isomerase), as well as those involved in the nonoxidative reactions of the PPP (e.g. epimerase, especially ribulose-5-phosphate 3-epimerse, transketolase (TKT), ribose-5-phosphate isomerase, fructose 1,6-bisphosphatase, and aldolase). In one preferred embodiment, modulation of the activity or level, or both, of TKT, is used for the method of the present invention.

Recent studies demonstrated that PPP is actively involved in the transcriptional glucose signaling pathway leading to activation of lipogenic genes (Doiron, et al., 1996; Massillon, 2001). Xylulose 5-phosphate (Xu-5-P), a major product of pentose phosphate pathway, was shown to be the glucose signaling molecule that positively regulates fat synthesis in the liver by activating the ChREBP transcription factor, a positive transcriptional inducer of the L-type pyruvate kinase gene and lipogenic enzyme genes, such as sterol regulatory element-binding protein-1c, acetyl-CoA carboxylases, and fatty acid synthase (Kabashima, et al., 2003; Towle, et al., 1997).

Therefore, in one embodiment, the methods and compositions of the present invention can control the level or activity of Xu-5-P as both acute and long-term regulation of glucose metabolism and fat synthesis. In addition, the activity or level of L-type pyruvate kinase gene and lipogenic enzymes, such as sterol regulatory element-binding protein-1c, acetyl-CoA carboxylases, and fatty acid synthase can also be modulated.

According to a preferred embodiment of the present invention, modulating the levels and/or activities of the transketolase is used to treat or prevent overweight or obesity. TKT is expressed in most tissues throughout the animal and plant kingdoms (Schenk et al., 1997, Molecular evolutionary analysis of the thiamine-diphosphate-dependent enzyme, transketolase. J. Mol. Evol. 44:552-572). It is prevalent in proliferating tumor cells, and its repression can inhibit tumor cell growth (Cascante et al., 2000, Role of thiamin vitamin B-1 and transketolase in tumor cell proliferation. Nutr. Cancer 36:150-154). As shown in the diagram below, transketolase is a key regulatory enzyme in the pentose phosphate pathway that provides ribose-5-phosphate and NADPH for nucleic acid synthesis and reductive biosynthesis, such as for de novo lipogenesis (Wood, 1985; Schenk, et al., 1998).

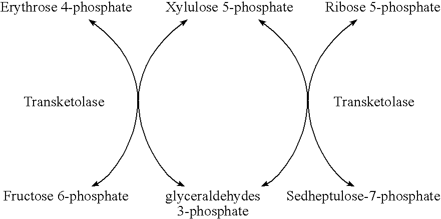

In non-oxidative pentose phosphate pathway, TKT enzyme reactions are directly involved in the production of Xu-5-P. By modulating the TKT enzyme activity, glucose mediated lipid metabolism can be modulated.

Many of the above identified enzymes are known and have been fully characterized, and the DNA sequences of genes encoding them are known. For example, Table 1 below lists some of the genes and their GenBank accession numbers. These numbers are gateways to the scientific literature on these enzymes' biological, biochemical, biophysical and genetic characteristics. All information contained therein are known to those skilled in the art and are specifically incorporated herein by reference.

The levels or activities of the enzymes (target protein) or the genes (target genes) that encoding these proteins or enzymes can be controlled by many methods known in the art. The present invention provides methods and compositions comprising antagonizing agents that inhibit, antagonize or reduce the levels or biological activities of the target genes or target proteins or both. In one embodiment, this invention provides neutralizing antibodies to the target proteins to inhibit their biological activities. In another embodiment of the invention, the antagonizing agents are antisense oligonucleotides. The antisense oligonucleotides preferably inhibit target gene expression by inhibiting translation of the target protein. In a further embodiment, the antagonizing agent is small interfering RNAs (siRNA, also known as RNAi, or RNA interference nucleic acids). siRNA are double-stranded RNA molecules, typically 21 n.t. in length, that are homologous to the target gene and interfere with the target gene's activity. In general, the preferred target of the treatment method of the present invention, and the delivery target of compositions of the present invention, are tissues or organs that are responsible for fat synthesis or where PPP is especially active.

TABLE 1
Examples of Enzymes Involved in the Pentose Phosphate
Pathway and Their Database Accession Numbers

| Enzyme Name | UniGene Name | Accession Number |
|---|---|---|
| Transketolase | Hs.89643 | NM_001064 |
| Ribulose-5-phosphate 3-epimerase | Hs.282260 | NM_006916 |
| Ribose-5-phosphate isomerase | Hs.79886 | NM_144563 |
| Aldolase A | Hs.273415 | NM_184043 |
| Aldolase B | Hs.315235 | NM_000035 |
| Fructose 1,6-bisphosphatase 1 | Hs.360509 | NM_000507 |
| Fructose 1,6-bisphosphatase 2 | Hs.61255 | NM_003837 |

An antibody suitable for the present invention may be a polyclonal antibody. Preferably, the antibody is a monoclonal antibody. The antibody may also be isoform-specific. The monoclonal antibody or binding fragment thereof of the invention may be Fab fragments, F(ab)2 fragments, Fab′ fragments, F(ab′)2 fragments, Fd fragments, Fd′ fragments or Fv fragments. Domain antibodies (dAbs) (for review, see Holt et al., 2003, Trends in Biotechnology 21:484-490) are also suitable for the methods of the present invention.

Once an antigen is known or available, various methods of producing antibodies against the antigen are well-known to those ordinarily skilled in the art (see for example, Harlow and Lane, 1988, Antibodies: A Laboratory Manual. Cold Spring Harbor Laboratory Press, Cold Spring Harbor, N.Y.; see also WO 01/25437). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In accordance with the present invention, the antibodies or binding fragments thereof may be characterized as those which are capable of specific binding to a target protein or an antigenic fragment thereof, preferably an epitope recognized by an antibody when the antibody is administered in vivo. Antibodies can be elicited in an animal host by immunization with a target protein-derived immunogenic component, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

The antibodies may be from humans, or from animals other than humans, preferably mammals, such as rat, mouse, guinea pig, rabbit, goat, sheep, and pig. Preferred are mouse monoclonal antibodies and antigen-binding fragments or portions thereof. In addition, chimeric antibodies and hybrid antibodies are embraced by the present invention. Techniques for the production of chimeric antibodies are described in e.g. Morrison et al., 1984, Proc. Natl. Acad. Sci. USA, 81:6851-6855; Neuberger et al., 1984, Nature, 312:604-608; and Takeda et al., 1985, Nature, 314:452-454.

Further, single chain antibodies are also suitable for the present invention (e.g., U.S. Pat. Nos. 5,476,786 and 5,132,405 to Huston; Huston et al., 1988, Proc. Natl. Acad. Sci. USA, 85:5879-5883; U.S. Pat. No. 4,946,778 to Ladner et al.; Bird, 1988, Science, 242:423-426 and Ward et al., 1989, Nature, 334:544-546). Single chain antibodies are formed by linking the heavy and light immunoglobulin chain fragments of the Fv region via an amino acid bridge, resulting in a single chain polypeptide. Univalent antibodies are also embraced by the present invention.

Many routes of delivery are known to the skilled artisan for delivery of anti-target antibodies. For example, direct injection may be suitable for delivering the antibody to the site of interest. It is also possible to utilize liposomes with antibodies in their membranes to specifically deliver the liposome to the area of the tumor where target gene expression or function is to be inhibited. These liposomes can be produced such that they contain, in addition to monoclonal antibody, other therapeutic agents, such as those described above, which would then be released at the tumor site (e.g., Wolff et al., 1984, Biochem. et Biophys. Acta, 802:259).

This invention also provides antisense nucleic acid molecules and compositions comprising such antisense molecules. The constitutive expression of antisense RNA in cells has been known to inhibit the gene expression, possibly via the blockage of translation or prevention of splicing. Interference with splicing allows the use of intron sequences which should be less conserved and therefore result in greater specificity, inhibiting expression of a gene product of one species but not its homologue in another species.

The term antisense component corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vivo conditions. This antisense molecule must have sufficient complementarity, about 18-30 nucleotides in length, to the target gene so that the antisense RNA can hybridize to the target gene (or mRNA) and inhibit target gene expression regardless of whether the action is at the level of splicing, transcription, or translation. The antisense components of the present invention may be hybridizable to any of several portions of the target cDNA, including the coding sequence, 3′ or 5′ untranslated regions, or other intronic sequences, or to target mRNA.

Antisense RNA is delivered to a cell by transformation or transfection via a vector, including retroviral vectors and plasmids, into which has been placed DNA encoding the antisense RNA with the appropriate regulatory sequences including a promoter to result in expression of the antisense RNA in a host cell. In one embodiment, stable transfection and constitutive expression of vectors containing target cDNA fragments in the antisense orientation are achieved, or such expression may be under the control of tissue or development-specific promoters. Delivery can also be achieved by liposomes.

For in vivo therapy, the currently preferred method is direct delivery of antisense oligonucleotides, instead of stable transfection of an antisense cDNA fragment constructed into an expression vector. Antisense oligonucleotides having a size of 15-30 bases in length and with sequences hybridizable to any of several portions of the target cDNA, including the coding sequence, 3′ or 5′ untranslated regions, or other intronic sequences, or to target mRNA, are preferred. Sequences for the antisense oligonucleotides to target are preferably selected as being the ones that have the most potent antisense effects. Factors that govern a target site for the antisense oligonucleotide sequence include the length of the oligonucleotide, binding affinity, and accessibility of the target sequence. Sequences may be screened in vitro for potency of their antisense activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides.

The preferred target antisense oligonucleotides are those oligonucleotides which are stable, have a high resistance to nucleases, possess suitable pharmacokinetics to allow them to traffic to target tissue site at non-toxic doses, and have the ability to cross through plasma membranes.

Phosphorothioate antisense oligonucleotides may be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNase H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

The delivery route will be the one that provides the best antisense effect as measured according to the criteria described above. In certain tumor related treatments, in vitro cell culture assays and in vivo assays using antisense oligonucleotides have shown that delivery mediated by cationic liposomes, by retroviral vectors and direct delivery are efficient. Another possible delivery mode is targeting using antibody to cell surface markers for the target cells. Antibody to target or to its receptor may serve this purpose.

Alternatively, nucleic acid sequences which inhibit or interfere with gene expression (e.g., siRNA, ribozymes, aptamers) can be used to inhibit or interfere with the activity of RNA or DNA encoding a target protein.
siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′, 5′ oligoadenylate synthetase (2′,5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48; Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411:428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerheadribozyme compositions of the invention (see below).

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:863-74). The effectiveness of the siRNA may be assessed by any of a number of assays following introduction of the dsRNAs. These include Western blot analysis using antibodies which recognize the target gene product following sufficient time for turnover of the endogenous pool after new protein synthesis is repressed, reverse transcriptase polymerase chain reaction and Northern blot analysis to determine the level of existing target mRNA.

Further compositions, methods and applications of siRNA technology are provided in U.S. patent application Nos. 6,278,039, 5,723,750 and 5,244,805, which are incorporated herein by reference.

Ribozymes are enzymatic RNA molecules capable of catalyzing specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach, 1988, Nature 334:585-591; and PCT Application. No. WO89/05852, the contents of which are incorporated herein by reference. Hammerhead ribozyme sequences can be embedded in a stable RNA such as a transfer RNA (tRNA) to increase cleavage efficiency in vivo (Perriman et al., 1995, Proc. Natl. Acad. Sci. USA, 92:6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al., 1998, Nature 393:284-9; Kuwabara et al., 1998, Nature Biotechnol. 16:961-5; and Kuwabara et al., 1998, Mol. Cell 2:617-27; Koseki et al., 1999, J. Virol 73:1868-77; Kuwabara et al., 1999, Proc. Natl. Acad. Sci. USA, 96:1886-91; Tanabe et al., 2000, Nature 406:473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

The ribozymes of the present invention also include RNA endoribonucleases (“Cech-type ribozymes”) such as the one which occurs naturally in Tetrahymena thermophila (known as the IVS, or L-19 IVS RNA) and which has been extensively described in Zaug, et al., 1984, Science, 224:574-578; Zaug, et al., 1986, Science 231:470-475; Zaug, et al., 1986, Nature 324:429-433; published International patent application No. WO88/04300; and Been, et al., 1986, Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or siRNA are substantially the same sequence of at least 5 and preferably 6, 7, 8, 9, 10, 11, 12, 13, 14, 15, 16, 17, 18, 19 or 20 or more contiguous nucleotides of a target nucleic acid.

In a long target RNA chain, significant numbers of target sites are not accessible to the ribozyme because they are hidden within secondary or tertiary structures (Birikh et al., 1997, Eur. J. Biochem. 245:1-16). To overcome the problem of target RNA accessibility, computer generated predictions of secondary structure are typically used to identify targets that are most likely to be single-stranded or have an “open” configuration (see Jaeger et al., 1989, Methods Enzymol. 183:281-306). Other approaches utilize a systematic approach to predicting secondary structure which involves assessing a huge number of candidate hybridizing oligonucleotides molecules (see Milner et al., 1997, Nat. Biotechnol. 15: 537-41; and Patzel and Sczakiel, 1998, Nat. Biotechnol. 16:64-8). Additionally, U.S. Pat. No. 6,251,588, the contents of which are herein incorporated by reference, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the siRNA oligonucleotides and ribozymes of the invention.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C., 1991, Anticancer Drug Des., 6:569-84; Helene, C., et al., 1992, Ann. N.Y. Acad. Sci., 660:27-36; and Maher, L. J., 1992, Bioassays 14:807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of target gene. DNA enzymes incorporate some of the mechanistic features of both antisense and ribozyme technologies. DNA enzymes are designed so that they recognize a particular target nucleic acid sequence, much like an antisense oligonucleotide. They are, however, catalytic and specifically cleave the target nucleic acid.

There are currently two basic types of DNA enzymes, both of which were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo are similar methods of delivery RNA ribozyme, as outlined in detail above. Additionally, one of skill in the art will recognize that, like antisense oligonucleotide, DNA enzymes can be optionally modified to improve stability and improve resistance to degradation.

The dosage ranges for the administration of the antagonists of the invention are those large enough to produce the desired effect. The dosage should not be so large as to cause adverse side effects, such as unwanted cross-reactions, anaphylactic reactions, and the like. Generally, the dosage will vary with the age, condition, sex and extent of disease of the patient and can be determined by one of skill in the art. The dosage can be adjusted by the individual physician in the event of any complication.

The present invention further provides small molecule inhibitors or antagonists of the target proteins or enzymes, many of which are known for the various target proteins. For example, many inhibitors of transketolase are known, such as 2′-methoxy-thiamin, pyrithiamine and dehydroepiandrosterone.

The antagonists of the invention can be administered parenterally by injection or by gradual perfusion over time. The antagonists can be administered intravenously, intraperitoneally, intramuscularly, subcutaneously, intracavity, or transdermally.

Another embodiment of the present invention relates to pharmaceutical compositions comprising one or more antagonists, according to the invention, together with a physiologically- and/or pharmaceutically-acceptable carrier, excipient, or diluent. Physiologically acceptable carriers, excipients, or stabilizers are known to those skilled in the art (see Remington's Pharmaceutical Sciences, 17th edition, (Ed.) A. Osol, Mack Publishing Company, Easton, Pa., 1985). Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

### EXAMPLES

### Example 1

### Treatment with a TKT Inhibitor Prevents Obesity in Mice

Adult mice fed with a high fat diet showed normal growth when treated with a TKT inhibitor, while the control group without inhibitor became obese. Increased TKT enzyme activity was observed in mice fed with a high fat diet, indicating that increased TKT enzyme activity could contribute to the overproduced body fat.

Oxythiamine (OT) has been widely used as a TKT inhibitor to study the relationship between PPP and tumor proliferation process in cultured tumor cell lines and in vivo.

The histotoxicity of OT was checked on some vital organs such as the liver, heart and kidney of mice hosting Ehrlich's tumor. These mice were treated with a dose of 400 mg/kg/day of OT. No signs of toxicity were observed in all these tissue as compared to the controls.

D12492 is a very high fat containing diet (with 60 kcal % fat), and has been widely used to induce obesity in rodents. The composition of this diet is shown in Table 2 below.

TABLE 2
Composition of D12492 Diet

| | | |
|---|---|---|
| Casein, 80 mesh | 200 | g |
| L-Cystine | 3 | g |
| Maltodextrin 10 | 125 | g |
| Sucrose | 68.8 | g |
| Cellulose, BW200 | 50 | g |
| Soybean oil | 25 | g |
| Lard | 245 | g |
| Mineral Mix S10026 | 10 | g |
| Dicalcium phosphate | 13 | g |
| Calcium carbonate | 5.5 | g |
| Potassium citrate, 1 H₂O | 16.5 | g |
| Vitamin Mix V10001 | 10 | g |
| Choline bitartrate | 2 | g |
| FD&C blue dye #1 | 0.05 | g |

A dose of 10-20 mg/kg/day of OT was administered orally to C57BL/6 mice fed with D12492. Diet-induced obesity was not developed in mice treated with OT. In contrast, the control mice developed obesity. We found that OT has a dosage dependent effects on preventing weight gain of mice fed with D112492.

The experimental mice fed with D12492 plus OT had normal growth rate comparing to the same age mice fed with a standard diet.

4 groups of C57BL/6 mice (5 mice each group) were used to test the dosage dependence of oxythiamine on weight control. The negative control was fed a regular diet, and positive group was fed a high fat diet only. Test groups were fed high fat diet and each orally administered with OT at a concentration of 2 mg/kg/day or 10 mg/kg/day.

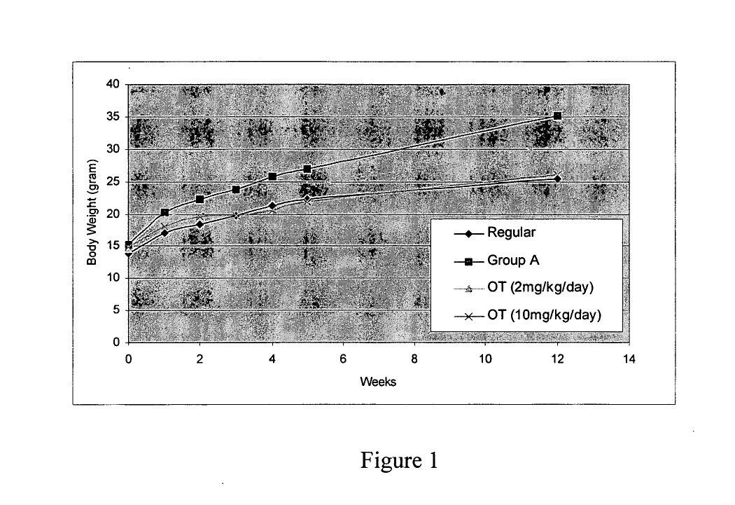

As shown in FIG. 1, and consistent with the results from the necropsy study of TKT heterozygotes (Xu et al., 2002), OT treated mice are near normal based on pathological studies.

A higher dosage of OT, 180 mg/kg/day, had a strong effect of weight loss on normal as well as fatty mice. Preferential reduction of fat mass was observed in OT fed mice. This is consistent with the result obtained from the published result of TKT haplo-insufficiency mice. The above results show that OT can be used as a potential anti-obesity drug to modulate body weight without any diet restriction.

### Example 2

### Restricted Supply of Thiamine Results in Preferential Reduction of Body Fat Mass in Mice

To test the effect of thiamine supplementation on TKT enzyme activity on adipose tissue, the AIN-93M diet (for composition, see Table 3 below), without thiamine, was used to feed C57BL/6 mice for two to three weeks in order to create thiamine insufficient conditions. As previously reported, continued feeding of animal with non-thiamine diet for three or more than three weeks will cause a thiamine deficiency disease similar to what observed in humans. (Kril, J. J. Metab Brain Dis. 11:9-17, 1996; Langlais et al., Metab Brain Dis. 11:19-37, 1996.) Thiamine concentration in vivo, especially regional aggregation of thiamine are known to stimulate TKT activity, thus PPP activity. Thiamine aggregation has been reported in tumor patients, especially with malignant tumors. Most actively synthesizing cells, such as tumor cells, have a strong ability to attract or aggregate thiamine.

TABLE 3
Composition of Diets Used
AIN-93M diet

| | |
|---|---|
| Cornstarch | 46.5692% |
| Casein | 14.0000% |
| Dextrinized cornstarch | 15.5% |
| Sucrose | 10.0% |
| Soybean oil | 4.0% |
| Alphacel, non-nutritive bulk | 5% |
| AIN-93M Mixture | 3.5% |
| L-Cystine | 0.18% |
| AIN-93-VX | 1.0% |
| Choline bitartrate | 0.25% |
| Tert-Butylhydroquinone | 0.008% |

AIN-93-VX (Vitamin mix)

| | | |
|---|---|---|
| Nicotinic acid | 3.00 | g/kg |
| D-Calcium pantothenate | 1.60 | g/kg |
| Pyridoxine HCl | 0.70 | g/kg |
| Thiamine HCl | 0.60 | g/kg |
| Riboflavin | 0.60 | g/kg |
| Folic acid | 0.20 | g/kg |
| D-Biotin | 0.02 | g/kg |
| Vitamin B₁₂ | 2.5 | g/kg |
| Alpha Tocophaerol powder (250 U/g) | 30.00 | g/kg |
| Vitamin A polmitate (250,000 U/g) | 1.60 | g/kg |
| Vitamin D₃ (4000,000 U/g) | 0.25 | g/kg |
| Phylloquinone | 0.075 | g/kg |
| Powdered sucrose | 959.655 | g/kg |

We found that restricted supply of thiamine for up to two weeks resulted in preferential reduction of body fat mass in mice. Reduced TKT enzyme activity was observed in mice fed with non-thiamine diet but not in mice with normal diet (AIN-93M). Mice fed with non-thiamine diet for two weeks were very healthy, major organs and tissues are generally normal based on the pathological study. Our study indicated that thiamine supply in diet had a direct relationship with TKT enzyme activity, thus affecting the fat synthesis.

Also used is an AIN-93M diet without thiamine, which is the AIN-93-VX used without Thiamine HCl.

### Example 3

### FWGE as TKT Inhibitor Reduces Body Weight Gain in Mice

Fermented wheat germ extract (FWGE) is nontoxic, strongly inhibits the PPP, and sometimes has been used as a supplement with other cancer therapies (Garami et al., J. Pediatr. Hemotol. Oncol., 26, 631-635, 2004). Our results and published data have revealed that it has TKT inhibitory activity (Comin-Anduix et al., J. Biol. Chem. 277, 46408-46414, 2002). Our initial results also indicated that mice treated with OT as low as 2 mg/kg/day can reduce the progression of obesity development significantly. In contrast, 100-400 mg/kg of OT has been successfully used to treat tumors in animals (Boros et al., Cance Res., 57, 4242-4248, 1997; Rais et al., FEBS Lett., 456, 113-118, 1999). These data indicate that products having mild activity, particularly natural or formulated plants and foods, might be effective enough for obesity prevention. The preliminary data suggest that FWGE should inhibit TKT adequately to show an effect. The ability to regulate TKT and the adaptive glucose regulatory pathway should provide further incentive to explore the consequences of safe TKT inhibition or preferential modulation of pentose phosphate pathway activity in certain tissues.

A dose of 0.356 g/kg/day of FWGE was administered to C57BL/6 mice fed with D12451 (Research Diets Inc., NJ). Diet-induced obesity was not observed in mice treated with FWGE. In contrast, mice fed with D12451 alone developed obesity or gained more weight than mice fed with a normal diet (D12450).

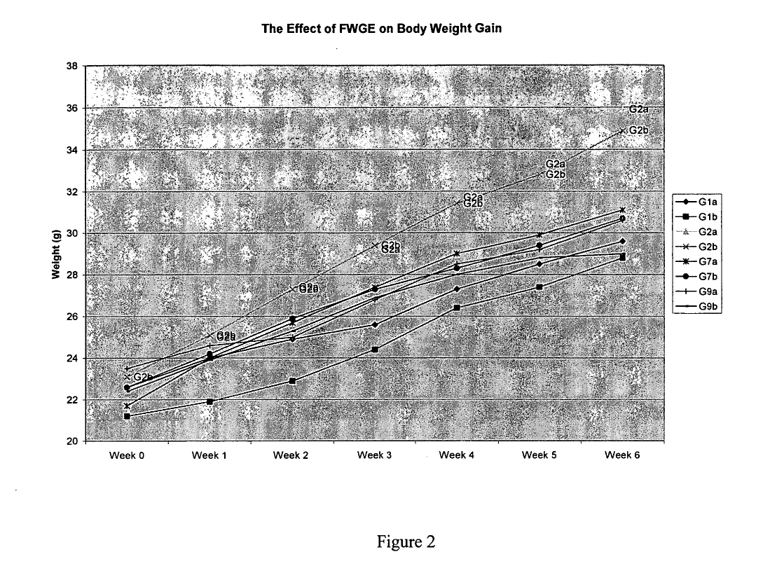

Mice fed with normal diet plus FWGE are healthy. No growth rate change observed in mice fed with normal diet alone and mice fed with normal diet plus FWGE (FIG. 2).

4 groups of C57BL/6 mice (10 mice each group) were used in the feeding experiments.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

### REFERENCES

1. Kopelman, P. G. Obesity as a medical problem. Nature. 404:635-643, 2000.
2. Spiegelman, B. M., Flier, J. S. Obesity and the regulation of energy balance. Cell 104:531-543, 2001.
3. Jequier, E. Pathways to obesity. Int J Obes Relat Metab Disord. Suppl 2:S 12-17, 2002.
4. Swinburn, B and Egger, G. Preventive strategies against weight gain and obesity. Obes Rev. 3:289-301, 2002
5. Hill, J. O., Melanson, E. L., Wyatt, H. T. Dietary fat intake and regulation of energy balance: implications for obesity. J Nutr. 130(2S Suppl):284S-288S, 2000.
6. Peters, J. C. Dietary fat and body weight control. Lipids. 38:123-127, 2003.
7. Masuzaki, H., et al. Transgenic Model of Visceral Obesity and the Metabolic Syndrome. Science. 294:2166-2170, 2001.
8. Willett, W. C. Dietary fat plays a major role in obesity: no. Obes Rev. 3:59-68, 2002.
9. Centers for Disease Control and Prevention (CDC). Trends in intake of energy and macronutrients—United States, 1971-2000.
10. MMWR Morb Mortal Wkly Rep. 53:80-82, 2004.
11. Minehira, K., Bettschart, V., Vidal, H., Vega, N., Di Vetta V, Rey V, Schneiter P, Tappy L. Effect of carbohydrate overfeeding on whole body and adipose tissue metabolism in humans. Obes Res. 11: 1096-1103, 2003.
12. Wood, T. The Pentose Phosphate Pathway, Academic Press/Harcourt Brace Jovanovich, New York, 1985.
13. Schenk, G., Duggleby, R. G., Nixon, P. F. Properties and functions of the thiamin diphosphate dependent enzyme transketolase. Int. J. Biochem. & Cell Biol. 30:1297-1318, 1998.
14. Hems, D. A., Rath, E. A., Verrinder, T. R. Fatty acid synthesis in liver and adipose tissue of normal and genetically obese (ob/ob) mice during the 24-hour cycle. Biochem J 150:167-173, 1975.
15. Gandemier, G., Pascal, G., Durand, G. In vivo changes in the rates of total lipid and fatty acid synthesis in liver and white adipose tissues of male rats during postweaning growth. Int J. Biochem. 14:797-804, 1982.
16. Patel, M. S., Owen, O. E., Goldman, L. I., Hanson, R. W. Fatty acid synthesis by human adipose tissue. Metabolism. 24:161-73, 1975.
17. Guo, Z. K., Cella, L. K., Baum, C., Ravussin, E., Schoeller, D. A. De novo lipogenesis in adipose tissue of lean and obese women: application of deuterated water and isotope ratio mass spectrometry. Int J Obes Relat Metab Disord. 24:932-937, 2000.
18. Hellerstein, M. K., et al. Measurement of de novo hepatic lipogenesis in humans using stable isotopes. J Clin Invest. 87:1841-1852, 1991.
19. Larsen, L. O. The role of de novo lipogenesis in development of obesity in man. Br J Nutr. 88:331-332, 2002;
20. Diraison, F., Dusserre, E., Vidal, H., Sothier, M., Beylot, M. Increased hepatic lipogenesis but decreased expression of lipogenic gene in adipose tissue in human obesity. Am J Physiol Endocrinol Metab. 282:E46-51, 2002.
21. Levine, A. S., Kotz, C. M., Gosnell, B. A. Sugars: hedonic aspects, neuroregulation, and energy balance. Am J Clin Nutr. 78:834S-842S, 2003.
22. Matsuo T, Baba Y, Hashiguchi M, Takeshita K, Izumori K, Suzuki H. Dietary D-psicose, a C-3 epimer of D-fructose, suppresses the activity of hepatic lipogenic enzymes in rats. Asia Pac J Clin Nutr. 10:233-237, 2001.
23. Foster G D, Wyatt H R, Hill J O, McGuckin B G, Brill C, Mohammed B S, Szapary P O, Rader D J, Edman J S, Klein S. A randomized trial of a low-carbohydrate diet for obesity. N Engl J. Med. 348:2082-2090, 2003.
24. Parks E J. Dietary carbohydrate's effects on lipogenesis and the relationship of lipogenesis to blood insulin and glucose concentrations. Br J Nutr. 87 Suppl 2:S247-253, 2002.
25. Doiron, B., Cuif, M. H., Chen, R., Kahn, A. Transcriptional glucose signaling through the glucose response element is mediated by the pentose phosphate pathway. J Biol. Chem. 271:5321-5324, 1996.
26. Massillon, D. Regulation of the glucose-6-phosphatase gene by glucose occurs by transcriptional and post-transcriptional mechanisms. Differential effect of glucose and xylitol. J Biol. Chem. 276:4055-4062, 2001.
27. Kril, J. J. Neuropathology of thiamine deficiency disorders. Metab Brain Dis. 11:9-17, 1996.
28. Langlais, P. J., Zhang, S. X., Savage, L. M. Neuropathology of thiamine deficiency: an update on the comparative analysis of human disorders and experimental models. Metab Brain Dis. 11:19-37, 1996.
29. Kabashima, T., Kawaguchi, T., Wadzinski, B. E., Uyeda, K. Xylulose 5-phosphate mediates glucose-induced lipogenesis by xylulose 5-phosphate-activated protein phosphatase in rat liver. Proc Natl Acad Sci USA. 100:5107-5112, 2003.
30. Towle, H. C., Kaytor, E. N., Shih, H. M. Regulation of the expression of lipogenic enzyme genes by carbohydrate. Annu Rev Nutr. 17:405-433, 1997.
31. Bell, N. H. Bone loss and gastric bypass surgery for morbid obesity. J Clin Endocrinol Metab. 89:1059-1060, 2004.
32. Brody, F. Minimally invasive surgery for morbid obesity. Cleve Clin J. Med. 71: 289, 293, 296-298, 2004.
33. Colquitt, J., Clegg, A., Sidhu, M., Royle, P. Surgery for morbid obesity. Cochrane Database Syst Rev. 2:CD003641, 2003.
34. Everhart, J. E. Contributions of obesity and weight loss to gallstone disease. Ann Intern Med. 119:1029-1035, 1993.
35. Glazer, G. Long-term pharmacotherapy of obesity 2000: a review of efficacy and safety. Arch Intern Med. 161:1814-1824, 2001.
36. Beermann, B., Melander, H., Sawe, J., Ulleryd, C., Dahlqvist, R. Incorrect use and limited weight reduction of orlistat (Xenical) in clinical practice. A cohort study. Eur J Clin Pharmacol. 57:309-311, 2001.
37. Hailer, C., Schwartz, J. B. Pharmacologic agents for weight reduction. J Gend Specif Med. 5:16-21, 2002.
38. Nightingale, S. From the Food and Drug Administration. JAMA. 276:14, 1996.
39. Bray, G. A. Use and abuse of appetite-suppressant drugs in the treatment of obesity. Annals of Internal Medicine 119:707-713, 1993.
40. Z.-P. Xu et al., Transketolase Haploinsufficiency Reduces Adipose Tissue and Female Fertility in Mice, Molecular and Cellular Biol. 22: 6142-6147, 2002.

What is claimed is:

1. A method for preventing or inhibiting overweight or obesity in a patient in need thereof, the method comprising inhibiting the activity of the pentose phosphate pathway (PPP) in said patient.

2. The method according to claim 1, wherein the activity or expression level of an enzyme involved in or related to the PPP is modulated.

3. The method according to claim 2, wherein the method comprising administering to the patient a pharmaceutical composition that inhibits the activity or expression level of the enzyme involved or related to PPP.

4. The method according to claim 2, wherein the enzyme involved in or related to PPP is a transketolase.

5. The method according to claim 3, wherein the pharmaceutical composition comprises a polynucleic acid molecule that specifically inhibits the expression of a gene encoding the enzyme, or a polypeptide that specifically inhibits the function of the enzyme.

6. The method according to claim 5, wherein the polynucleic acid molecule is a short interfering RNA molecule, or an antisense nucleic acid molecule that is specific against the gene, or a nucleic acid molecule that forms a triplex with the gene, thereby inhibiting the expression of the gene.

7. The method according to claim 5, wherein the polypeptide is an antibody against the gene or the enzyme.

8. The method according to claim 7, wherein the antibody is a monoclonal antibody, or an active fragment thereof.

9. The method according to claim 7, wherein the antibody is a humanized antibody.

10. A method according to claim 7, wherein the antibody is a human antibody.

11. The method according to claim 2, wherein the activity of the enzyme is inhibited by providing an analog of a substrate of the enzyme, an inactive analog of a co-factor of the enzyme, or a compound that binds to an active site of the enzyme that prevents the enzyme from binding to its substrate.

12. The method according to claim 2, wherein the activity of the enzyme is inhibited by limiting the availability to the enzyme a co-factor.

13. The method according to claim 12, wherein the enzyme is TKT and the co-factor is thiamine.

14. A pharmaceutical composition for treating or preventing overweight or obesity, comprising an antagonist to an enzyme involved or related to the pentose phosphate pathway, and a pharmaceutically acceptable excipient.

15. The pharmaceutical composition according to claim 14, wherein the antagonist is a polynucleotide molecule or a polypeptide molecule.

16. The pharmaceutical composition according to claim 15, wherein the polynucleotide molecule is short interfering RNA molecule, or an antisense nucleic acid molecule that is specific against the gene, or a nucleic acid molecule that forms a triplex with the gene, thereby inhibiting the expression of the gene.

17. The pharmaceutical composition according to claim 15, wherein the polypeptide molecule is an antibody against the enzyme.

18. The pharmaceutical composition according to claim 14, wherein the antagonist is a small molecule.

19. The pharmaceutical composition according to claim 18, wherein the enzyme is TKT and the small molecule is oxythiamine.

20. The pharmaceutical composition according to claim 14, wherein the enzyme is TKT and the antagonist is an bioactive composition from a preparation of fermented wheat germ.

21. A method for screening for a substance that inhibits the expression of a gene encoding a transketolase (a TKT gene), the method comprising: (1) providing a candidate substance to be tested; (2) applying said candidate substance to a cell expressing a TKT gene or a recombinant TKT gene construct, (3) detecting the level of expression of the TKT gene or TKT gene construct in the presence of the candidate substance, and (4) determining a level of expression of the TKT in the absence of the candidate substance, wherein a substance that decreases the level of expression of the TKT gene is selected.

22. A method for screening for a substance that inhibits the activity of transketolase (TKT), the method comprising: (1) providing a candidate substance to be tested; (2) applying said candidate substance to a preparation of TKT, (3) detecting the level of activity of TKT in the presence of the candidate substance, and (4) determining a level of activity of TKT in the absence of the candidate substance, wherein a substance that decreases the level of activity of TKT gene is selected.
